(19) United States
(12) Patent Application Publication
(10) Pub. No.: US 2003/0049663 A1
(43) Pub. Date: Mar. 13, 2003

(54) Use of reflections of DNA for genetic analysis

(76) Inventors: Michael Wigler, Cold Spring Harbor, NY (US); Robert Lucito, East Meadow, NY (US); Lidia Serina, Hamden, CT (US); Eli Hatchwell, Cold Spring Harbor, NY (US)

Correspondence Address: PENNIE AND EDMONDS, 1155 AVENUE OF THE AMERICAS, NEW YORK, NY, 100362711

(21) Appl. No.: 10/183,695

(22) Filed: Jun. 27, 2002

Related U.S. Application Data
(60) Provisional application No. 60/301,192, filed on Jun. 27, 2001.

(51) Int. Cl. C12Q 1/68; C07H 21/04; C12P 19/34

(52) U.S. Cl. 435/6; 435/91.2; 536/23.1

(57) ABSTRACT

The present invention to provides a solution to problems associated with the use of hybridization for genetic analysis, including but not limited to the use of microarray technology for the analysis DNA. The present invention provides compositions and methods for the use reflections of DNA in genetic analysis. The present invention is also directed to methods for the production of reflections of DNA.

This application claims priority of U.S. Provisional Application No. 60/301,193, filed Jun. 27, 2001, incorporated herein in its entirety.

This invention was made with Government support under Contract No. R33-CA81674-03 awarded by the National Institutes of Health. The Government has certain rights to this invention.

### 1. FIELD OF THE INVENTION

The field of the invention is genetic analysis.

### 2. BACKGROUND OF THE INVENTION

Citation or identification of any reference in Section 2 or any section of this application shall not be construed as an admission that such reference is available as prior art to the present invention.

2.1. Microarray Technology

Although global methods for genomic analysis, such as karyotyping, determination of ploidy, and more recently comparative genomic hybridizaton (CGH) (Feder et al., 1998, Cancer Genet. Cytogenet. 102:25-31; Gebhart et al., 1998, Int. J. Oncol. 12:1151-1155; Larramendy et al., 1997, Am. J. Pathol. 151:1153-1161; Lu et al., 1997, Genes Chromosomes Cancer 20:275-281, all of which are incorporated herein by reference) have provided useful insights into the pathophysiology of cancer and other diseases or conditions with a genetic component, and in some instances have aided diagnosis, prognosis and selection of treatment, current methods do not afford a level of resolution of greater than can be achieved by standard microscopy, or about 5-10 megabases. Moreover, while many particular genes that are prone to mutation can be used as probes to interrogate the genome in very specific ways (Ford et al., 1998, Am. J. Hum. Genet. 62:676-689; Gebhart et al., 1998, Int. J. Oncol. 12:1151-1155; Hacia et al., 1996, Nat. Genet. 14:441-447, all of which are incorporated herein by reference), this one-by-one query is an inefficient and incomplete method for genetically typing cells.

With the advent of microarray, or “chip” technology, it is now clearly possible to contemplate obtaining a high resolution global image of genetic changes in cells. Two general approaches can be conceived. One is to profile the expression pattern of the cell using microarrays of cDNA probes (DeRisi et al., 1996, Nat. Genet. 14:457-460). This method is very likely to yield useful information about cancer, but suffers limitations. First, the interpretation of the data obtained and its correlation with disease process is likely to be a complex and difficult problem: multiple changes in gene expression will be observed that are not relevant to the disease of interest. Second, our present cDNA collections are not complete, and any chip is likely to be obsolete in the near future. Third, while a picture of the current state of the cell might be obtained, there would be little direct information about how the cell arrived at that state. Lastly, obtaining reliable mRNA from biopsies is likely to be a difficult problem, because RNA is very unstable and undergoes rapid degradation due to the presence of ubiquitous RNAses.

The second approach is to examine changes in the cancer genome itself. DNA is more stable than RNA, and can be obtained from poorly handled tissues, and even from fixed and archived biopsies. The genetic changes that occur in the cancer cell, if their cytogenetic location can be sufficiently resolved, can be correlated with known genes as the data bases of positionally mapped cDNAs mature. Thus, the information derived from such an analysis is not likely to become obsolete. The nature and number of genetic changes, can provide clues to the history of the cancer cell. Finally, a high resolution genomic analysis may lead to the discovery of new genes involved in the etiology of the disease or disorder of interest.

Microarrays typically have many different DNA molecules, often referred to as probes, fixed at defined coordinates, or addresses, on a flat, usually glass, support. Each address contains either many copies of a single DNA probe, or a mixture of different DNA probes, and each DNA molecule is usually 2000 nucleotides or less in length. The DNAs can be from many sources, including genomic DNA or cDNA, or can be synthesized oligonucleotides. For clarity and brevity, we refer to those chips with genomic or cDNA derived probes as DNA chips and those chips with synthesized oligonucleotide probes as oligo chips, respectively. Chips are typically hybridized to samples, applied as single stranded nucleic acids in solution.

The extent of hybridization with samples at a given address is determined by many-factors including the concentration of complementary sequences in the sample, the probe concentration, and the volume of sample from which each address is able to capture complementary sequences by hybridization. We refer to this volume as the diffusion volume. Because the diffusion volume, and hence, the potential hybridization signal, may vary from address to address in the hybridization chamber, the probe array is most accurate as a comparator, measuring the ratio of hybridization between two differently labeled specimens (the sample) that are thoroughly mixed and therefore share the same hybridization conditions, including the same diffusion volume. Typically the two specimens will be from diseased and disease free cells.

We distinguish between compound and simple DNA probe arrays based on the nucleotide complexity of the probes at each address. When this nucleotide complexity is less than or equal to about 1.2 kb per address, we speak of simple DNA probe arrays. When it exceeds 1.2 kb per address, we speak of compound probe arrays. Simple probe arrays are currently able to detect cDNA species that are present at 2 to 10 copies of mRNA per cell when contacted with a solution containing a total cDNA concentration of 1 mg/ml. The threshold of detection of a given species is estimated to be in the range of 4 to 20 ng/ml. Because a simple probe array is generally able to capture only a single species of DNA from the sample, this detection threshold poses a problem for the use of simple DNA probe arrays for analysis of genomic DNA. The concentration of a unique 700 bp fragment of human genomic DNA (which has a total complexity of about 3000 mb) in a solution of total genomic DNA dissolved at its maximum concentration of 8 mg/ml would be about 2 ng/ml, just below the lower estimate of the threshold of detection. Hence, in its unaltered format, the simple DNA probe chip would not suffice for the robust detection of genomic sequences.

The compound chip partially addresses this problem by increasing the nucleotide complexity of different probes at a given address, allowing for the capture of several species of DNA fragments at a single address. The signals of the different captured species combine to yield a detectable level of hybridization from genomic DNA. Present forms of compound probe arrays place the insert found in a single clone of a megacloning vector, such as a BAC, at each address. Because each address contains fragments derived from the entire BAC clone, several problems are created. The presence of repeat elements in the genomic inserts requires quenching with cold unlabeled DNA. Also, the great size of the megacloning vector inserts limits the positional resolution. For example, in the case of a compound probe array made of BACs, hybridization to a particular address reveals only to which BAC the hybridizing sequence is complementary, and does not reveal the specific complementary gene or sequence within that BAC. Another drawback is the presence of DNA derived from the megacloning vector and host sequences. The steps of excising and purifying the genomic DNA inserts from the vector and host sequences complicate and hinder rapid fabrication of microarrays.

2.2. Representations

A representation of DNA is a sampling of DNA, for example, the genome, produced by a restriction endonuclease digestion of genomic or other DNA, followed by linkage of adaptors and then amplification with primers complementary to the adaptors (Lucito et al., 1998, Proc. Natl. Acad. Sci. USA 95:4487-4492, International Patent Publication No. WO99/23256, each incorporated herein by reference). Generally, only fragments in the size range of 200-1200 bp amplify well, so the representation is a subset of the genome.

Representations can be made from very small amounts of starting material (e.g., from 5 ng of DNA), and are very reproducible. The reproducibility of representations has been demonstrated in several publications (Lisitsyn et al., 1995, Proc. Natl. Acad. Sci. USA 92:151; and Lucito et al., 1998, Proc. Natl. Acad. Sci. USA 95:4487-4492, both of which are incorporated herein by reference).

### 3. SUMMARY OF THE INVENTION

It is an object of the present invention to provide a solution to problems associated with genetic analysis, e.g., the use of microarray technology for the analysis DNA. The present invention provides compositions and methods for the use of reflections of DNA for hybridization, for example, in microarray technology.

For any previously described or future use of a microarray technology, including the use of representations in microarray technology, it is an object of the present invention to provide, prior to hybridization of the sample DNA to the microarrayed probe DNA, a reflection of the sample DNA to be hybridized to the array.

It is an object of the present invention to provide for the use of reflections of DNA in microarray technologies.

In one embodiment, the present invention provides compositions and methods for the use of reflections of simple and compound representations of DNA in microarray technology. A representation of DNA is a sampling of DNA produced by a restriction endonuclease digestion of genomic or other DNA, followed by linkage of adaptors and then amplification with primers complementary to the adaptors. The DNA may be from any source. Sources from which reflections can be made include, but are not limited to, genomic or cDNA from tumor biopsy samples, including breast cancer and prostate cancer biopsies, normal tissue samples, tumor cell lines, normal cell lines, cells stored as fixed specimens, autopsy samples, forensic samples, paleo-DNA samples, microdissected tissue samples, isolated nuclei, and fractionated cell or tissue samples. Optionally, the reflection can be prepared from a simple or compound representation.

The invention provides for the production of a reflection of DNA fragments comprising the steps of (a) contacting a first collection of DNA fragments with a second collection of DNA fragments under conditions such that hybridization between the first and second collections of DNA fragments can occur, thereby forming heteroduplex double stranded DNA fragments having one strand of DNA from the first collection of DNA fragments and a second strand of DNA from the second collection of DNA fragments; (b) purifying said heteroduplexes; (c) removing the second strands of DNA from the first strands; and (d) synthesizing double stranded DNA from the first strand using the first strand as a template.

A “reflection,” as used herein, is an enriched collection of DNA fragments that has been produced by enriching for fragments comprising those sequences present in a first collection of DNA fragments that are also present in a second collection of DNA fragments by hybridizing the first and second collections of DNA fragments together and selecting those DNA fragments in the first collection of DNA fragments that hybridize to fragments present in the second collection of DNA fragments. The collection of DNA fragments so selected is a “reflection.”

As used herein, the term “simple representation” refers to a sampling of DNA produced by a restriction endonuclease digestion of genomic or other DNA, followed by linkage of adaptors and then amplification with primers complementary to the adaptors.

As used herein, the term “compound representation” refers to a representation of a representation.

Reflections are useful for, but not limited to, mapping of complex genomes, detecting single nucleotide polymorphisms, determining gene copy number, deletion mapping, determining loss of heterozygosity, and comparative genomic hybridization.

### 4. BRIEF DESCRIPTION OF THE FIGS.

The present invention may be more fully understood by reference to the following detailed description of the invention, examples of specific embodiments of the invention and the appended FIGS. in which:

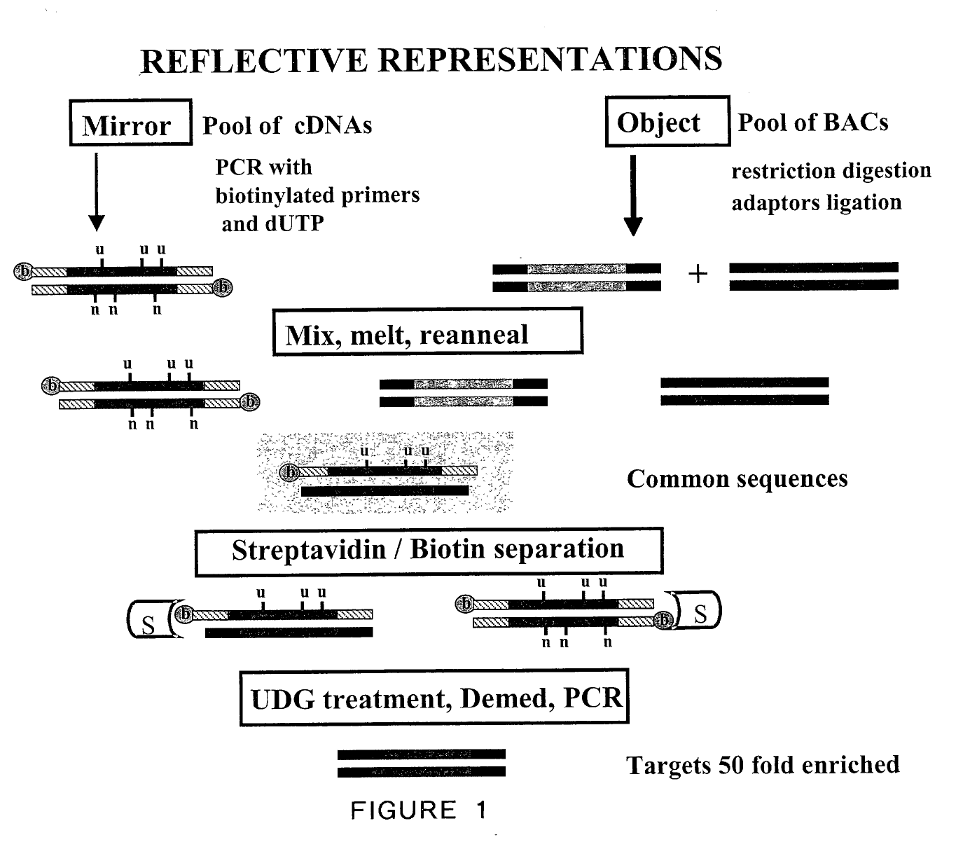

FIG. 1: Schematic of Reflective Representations. The “mirror” DNA (pool of cDNAs) is amplified using universal biotinylated M13 primers and dNTPs substituting 20% dUTP for dTTP. The “object” DNA (pool of BACs) is digested with Sau3AI and adaptors are ligated. Both DNAs are then mixed, melted and re-annealed. The expected targets are the heteroduplexes between one strand of mirror and one strand of object. To enrich the reflective representations with the targets or “object”, a streptavidin purification, Uracyl-DNA-glycosilase and Demed treatments are carried out on the fragments hybridized. A final PCR using the primers ligated to the object leads to the production of the reflective representation.

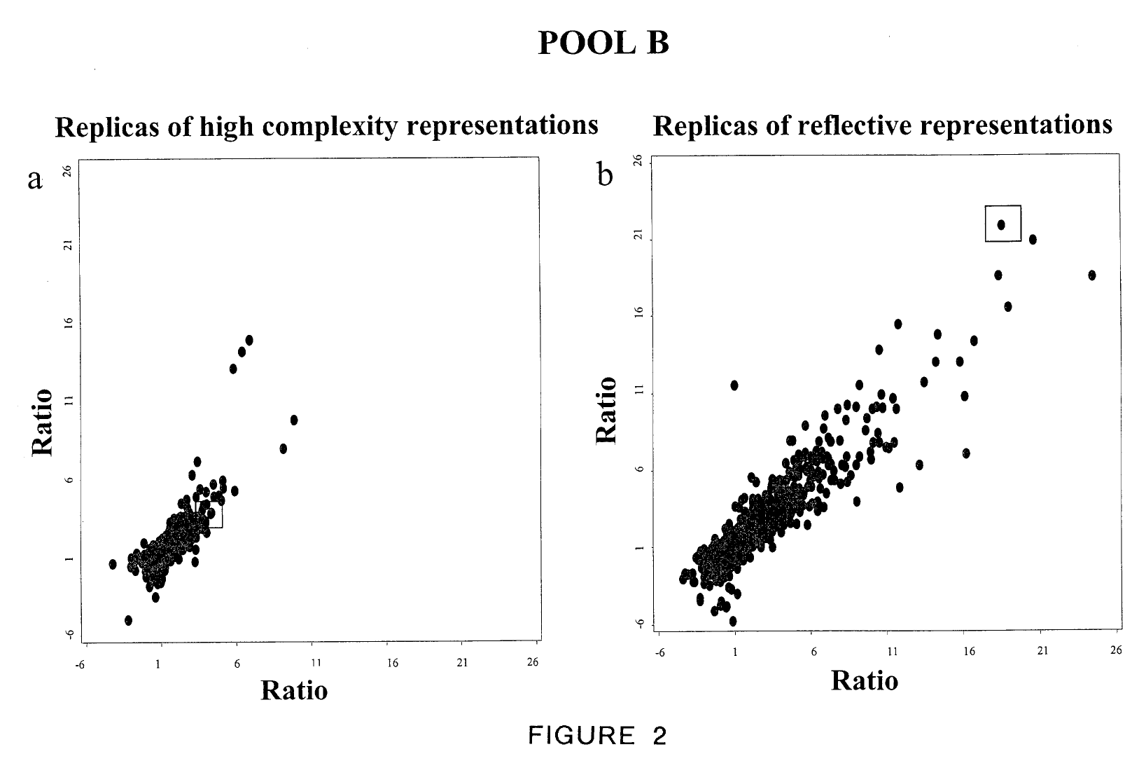

FIGS. 2A and 2B: Increase of the signal-to-noise ratio. Results of array hybridization where samples were compared to a universal reference or “denominator”. Resulting ratio calculated for one experiment is plotted against the ratios obtained from another experiment. Each experiment pair is an analysis of representations prepared in parallel. FIG. 2A represents the comparison of two Sau3AI representations prepared in parallel (unreflected representations) from BAC pool B after hybridizing the representations to the cDNA microarray. FIG. 2B represents the ratio of parallel reflective representations of BAC pool B in the same hybridization conditions. The spot outlined with a square demonstrates the increase of the ratio between unreflective representations (FIG. 2A) and reflective representation (FIG. 2B).

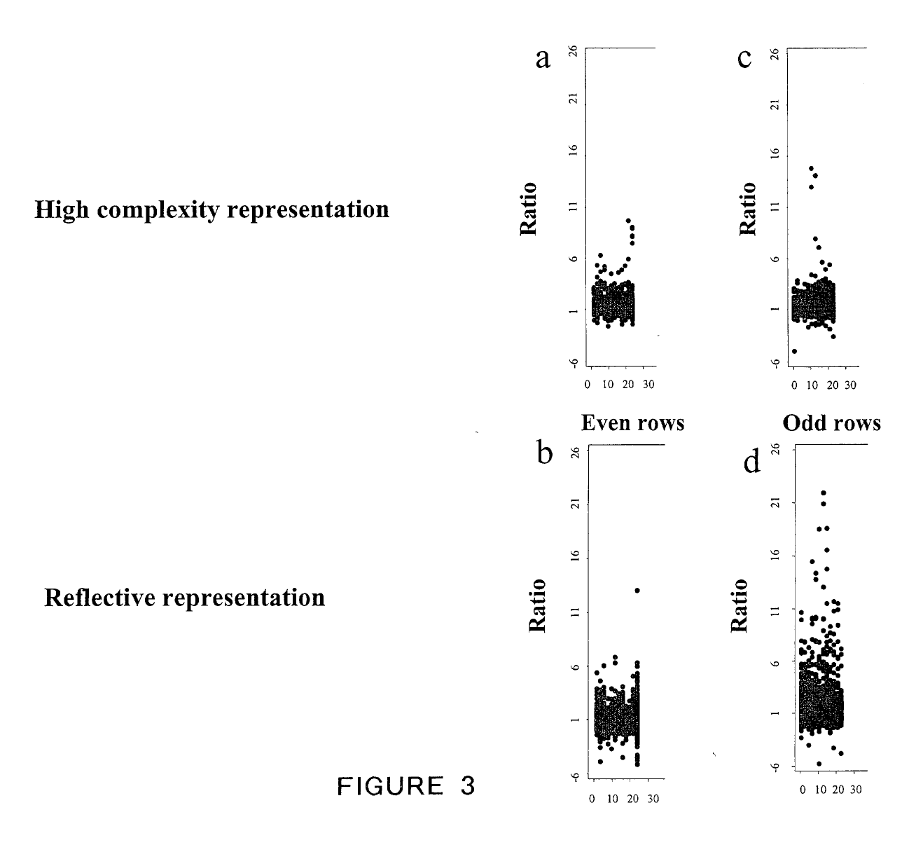

FIGS. 3A-3D: Reflective representations increase the signal-to-noise ratio of common probes between the “mirror” and the “object”. Experimental results are plotted such that the feature ratio is on the y-axis and the row location of the feature is plotted on the x-axis. A feature is a term used for the signal detected at a given address on the array. The feature ratio is the ratio of the two signals present at a given feature. FIGS. A and B represent the ratio of features printed in the even rows of the chip. Fragments in the even rows of the chip were absent in the “mirror” which is used to prepare the reflective representations. FIG. A is the result of a Sau3AI representation hybridized to the array and FIG. B, the results for a reflective representation. FIGS. C and D represent the ratio of features printed in the odd rows of the chip. Fragments in the odd rows of the chip were present in the “mirror” which is used to prepare the reflective representations. FIG. C shows the result of array hybridization with a Sau3AI representation and FIG. D, the results for a reflective representation.

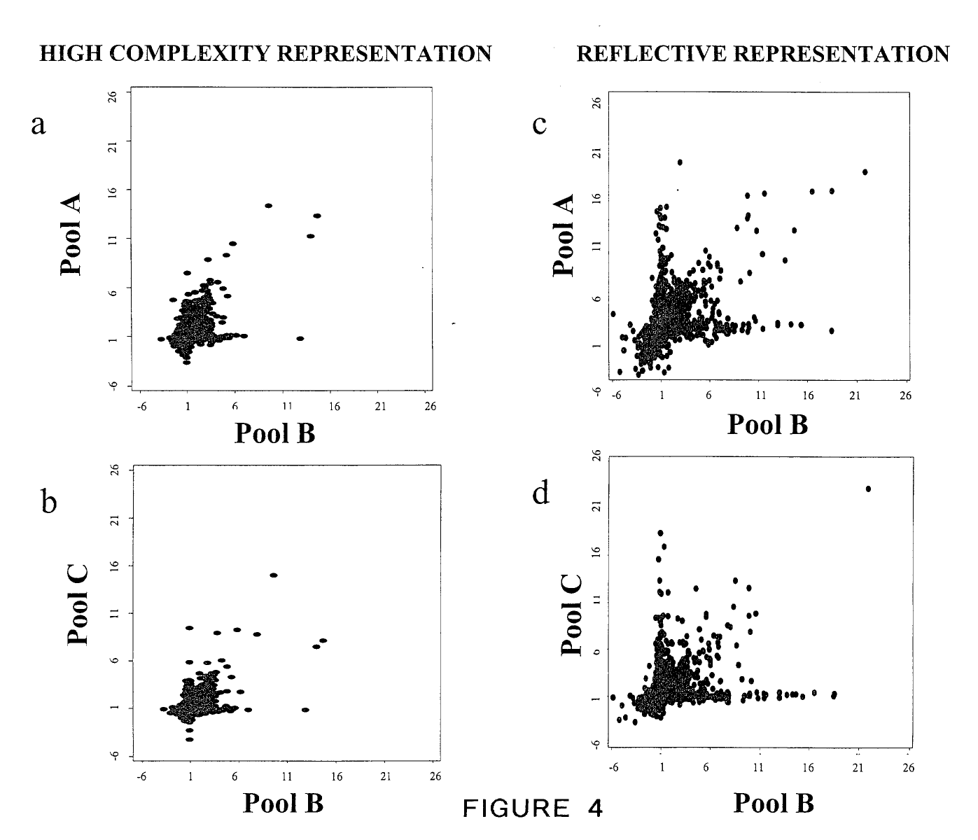

FIG. 4: Assignment of specific features or cDNAs to a BAC pool. Results of array hybridization plotted similarly to FIG. 1 except in this case feature ratios for reflection against one BAC pool as mirror is compared to reflection against another BAC pool as mirror. FIGS. A and B represent the ratios of unreflected representations (high complexity representations) hybridized to the array. FIG. A Sau3AI representation of BAC pool A compared to a Sau3AI representation of BAC pool B. and FIG. B Sau3AI representation of BAC pool C compared to a Sau3AI representation of BAC pool B. FIGS. C and D represent the ratios of reflective representations after hybridization. FIG. C comparison of a reflection of BAC pool A versus a reflection of BAC pool B and FIG. D as a comparison of a reflection of BAC pool C versus BAC pool B.

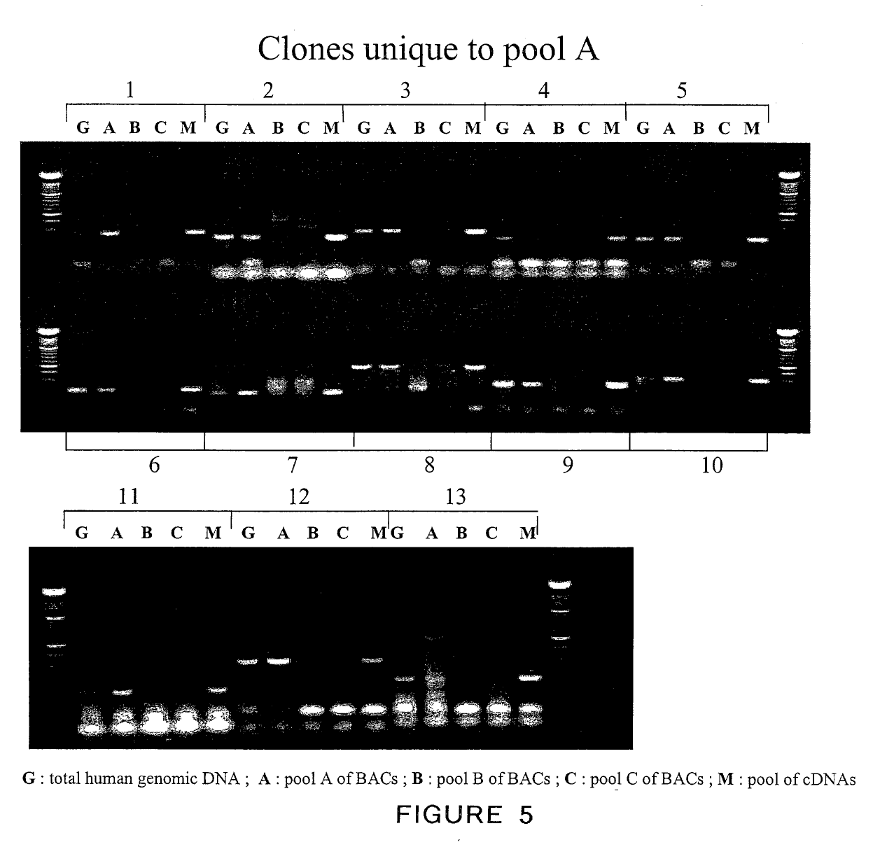

FIG. 5: Verification of the array experiments. Primer pairs have been designed from each cDNA having an elevated ratio after hybridization of reflective representation on the cDNA array. PCR has been performed on total human genomic DNA (G), BAC pool A (A), BAC pool B (B), BAC pool C (C), and the mirror (M). The figure represents the PCR results for cDNA chosen from the hybridization of a reflective representation from BAC pool A on the cDNA chip.

### 5. DETAILED DESCRIPTION OF THE INVENTION

The present invention provides for the use of reflections of DNA in genetic analysis, preferably microarray technology. The reflection may be prepared from any sample, including simple and compound representations of DNA. Representations are used to obtain a reproducible sampling of the genome that has reduced complexity.

The principle of this method is to use the collection of fragments, for example fragments arrayed in a microarray, to isolate the complimentary fragments from a sample for analysis. This creates a sample for hybridization that has a complexity on the order of the array being used for hybridization. By doing this the complexity of the sample can be dropped enormously. This in turn allows for better signal to noise for the probes on the array. This attribute allows the identification of specific fragments from genomes of size and complexity that could not normally be analyzed by conventional methods. The method of the invention can be used to analyze genome copy number in samples such as human genomic DNA compared on cDNA arrays. Reflection of normal and tumor DNA samples are compared to identify regions of the genome that undergo copy number fluctuation in cancer corresponding to the cDNAs or genes on the array.

Any use of a simple or compound representation as a source for the probe attached to a chip, or as the sample hybridized to the chip, or as DNA from which a probe to be hybridized to an array is derived, is within the scope of the invention. Arrays comprising probes derived from a representation by any method, for example by using the representation as a template for nucleic acid synthesis (e.g., nick translation, random primer reaction, transcription of RNA from represented DNA, oligonucleotide synthesis), or by manipulating the representation (e.g., size fractionation of the representation, gel purified fragments from the representation to the array) are also within the scope of the invention. Several applications of representations to DNA microarray technology are described below.

It is preferable that the one or more represented biological samples, and at least a fraction of the DNA comprising the microarray be from the same species. In a particular embodiment, the one or more samples are from a human, and at least a portion of the DNA on the microarray is human in origin. DNA from any species may be utilized according to the invention, including mammalian species (including but not limited to pig, mouse, rat, primate (e.g., human), dog and cat), species of fish, species of reptiles, species of plants and species of microorganisms.

5.1. Reflections

Any use of a reflection of DNA is within the scope of the present invention and several non-limiting examples are described below.

It is an object of the present invention to provide for the use of reflections of DNA in microarray technologies.

As explained above, a “reflection,” as used herein, is an enriched collection of DNA fragments that has been produced by enriching for fragments comprising those sequences present in a first collection of DNA fragments that are also present in a second collection of DNA fragments by hybridizing the first and second collections of DNA fragments together and selecting those DNA fragments in the first collection of DNA fragments that hybridize to fragments present in the second collection of DNA fragments. The collection of DNA fragments so selected is a “reflection.” The first collection of DNA fragments may be referred to as the “object,” and the second collection of DNA fragments may be referred to as the “mirror.” The selected fragments can then be amplified by any means known to one of skill in the art, for example, via the polymerase chain reaction. In a preferred embodiment, the first collection of DNA sequences is a sample DNA that is to be hybridized to a microarray, while the second collection of DNA sequences is comprised of the DNA probes on the microarray. The DNA to be reflected may be from any source, and, in a preferred embodiment, is a representation. A non-limiting description of the preparation of a reflection of a sample to be used for hybridization to probes present in a microarray follows.

First, the sample DNA is prepared. Sample DNA is digested with a restriction enzyme, e.g., Sau3Ai or BglIII. Preferably, adaptors are then ligated to the digested sample DNA. Optionally, the sample DNA can be amplified via PCR utilizing primers complementary to the adaptor sequences, creating a representation of the sample DNA.

Second, the probe DNA is prepared. Probe DNA is prepared such that 1) double stranded DNA containing at least one strand of probe DNA may be separated from double stranded DNA not containing a strand of probe DNA, and 2) probe DNA can be specifically removed from a mixture of sample DNA and probe DNA. This may be accomplished by, for example, amplifying probe DNA using a biotinylated primer to facilitate separation by using streptavidin, and incorporating uracil into the probe DNA, e.g., 20% of the thymidine residues in the probe DNA replaced with uracil residues, to facilitate removal through specific digestion of uracil containing sequences. Other methods of separating heteroduplexes include incorporating an oligonucleotide sequence into each probe DNA and using a sequence complementary thereto immobilized on a column to bind all DNAs incorporating such sequence. Other methods of removing probe DNA strands include incorporating methylated nucleic acids into the probe strand and utilizing a methylation sensitive restriction endonuclease, incorporating a thio-nucleotide on the end of the probe DNA to render it insensitive to a 3′ exonuclease, thereby causing the exonuclease to specifically degrade the probe DNA, or incorporating ribonucleotides into the probe DNA and specifically degrade the probe DNA strands by exposing them to alkaline conditions. Such methods are fully described in Cheung, V. G. and Nelson, S. F. (1998) Genomics 47, 1-6; Rys, P. N. and Persing, D. H. (1993) J. Clinical Microbio. 31, 2356-2360; Walder, R. Y., Hayes, J. R., and Walder, J. A. (1993) Nucleic Acids Res. 21, 4339-4343; and Zeng, J., Gorski, R. A., and Hamer, D. (1994) Nucleic Acids Res. 22, 4381-4385, each of which is incorporated by reference in its entirety.

Third, the probe DNA and the sample DNA are hybridized to one another. The probe DNA is mixed with the sample DNA in such ratio and under conditions that allow for hybridization of the probe DNA to the sample DNA, e.g., 13 hours at 65° C. Conditions of hybridization are known to those of skill in the art. Specific conditions can be found, for example, in a more detailed protocol in Lisitsyn, N. et al, (1993) Science 258, 946-951, which is incorporated herein by reference in its entirety. The probe DNA and sample DNA may be hybridized to one another in any ratio. Preferably, the probe DNA is in excess with respect to the DNA sequences present in the sample capable of hybridizing to the probe DNA sequences. Preferably, the probe DNA is in 5 fold excess, 10 fold excess, 100 fold excess, 500 fold excess, or 1000 fold excess.

Fourth, the sequences present in the sample DNA that hybridize to sequences present in the probe DNA are purified. Heteroduplexes (i.e., double stranded DNA consisting of one strand of probe DNA and one strand of sample DNA) are isolated from the hybridization reaction by, for example, using magnetic beads linked to streptavidin, which will specifically bind to any biotinylated sequences. Such a magnetic bead separation system is available from Promega. The probe DNA strand present in the heteroduplexes is then removed, by, for example, denaturing the double stranded DNA and specifically digesting the probe DNA. This can be accomplished by exposing the heteroduplexes to alkaline conditions, and, for probe DNA that has uracil residues incorporated therein, exposing the single stranded DNA molecules to a uracil-DNA glycosilase followed by a N,N′-dimethylethylene-diamine treatment to cut the DNA strands containing uracil.

The product of the above steps is the enrichment in the sample DNA of those sequences present in the sample DNA that can hybridize to sequences present in the probe DNA. A sample so enriched is termed a “reflection.” The probe DNA may be referred to as the “mirror,” and the original sample DNA may be referred to as the “object.” The resulting reflection may then be amplified by any means known to one of skill in the art, for example, via PCR, and used in hybridization procedures.

5.2. Uses of Reflections

The invention provides reflections hybridized to microarrays, and methods of producing such hybridized reflections. The invention also provides for the production of data in computer or otherwise readable form generated from hybridizations of reflections to microarrays.

Reflections may be utilized in any application where a sample DNA is to be hybridized to probe DNA, whether the probe DNA is present in an array or not. For example, sample DNA that is to be hybridized to probes in a microarray may be reflected using the members of the microarray as a mirror prior to hybridization to the array. Thus, any use of a microarray may be enhanced by utilizing a reflection of the sample DNA created using the probe sequences on the microarray as a mirror. For example, sample DNA reflections are suitable for microarray based mapping and detection of polymorphisms or change in gene copy number. Preferably, the sample DNA is a representation, or contains fragments from a representation or fragments derived from a representation. The making and use of representations is described in U.S. patent application Ser. No. 09/561,881, incorporated herein in its entirety. In a particular embodiment, the probe DNA is a representation, or consists of fragments present or derived from those in a representation. In an alternative embodiment, the probe DNA is not a representation and does not contain fragments from a representation or fragments derived from a representation. In an alternative embodiment, the sample DNA is not a representation and does not contain fragments from a representation or fragments derived from a representation. In a preferred embodiment, when both the sample DNA and the probe DNA are from a representation, or contain fragments from a representation or fragments derived from a representation, both the sample and probe representations are prepared the same way, i.e., using the same one or more restriction endonucleases, adaptors and PCR primers.

The method of the invention can be used to analyze genome copy number in samples such as human genomic DNA compared on cDNA arrays. Reflections of DNA from normal and tumor cells or tissue can be compared to identify regions of the genome that undergo copy number fluctuation in cancer corresponding to the cDNAs or genes found on the array.

The reflected representations of the invention may be used for analysis of single nucleotide polymorphisms (SNPs). The fragments on the array need not be identical to those used in the mirror. For example, in one embodiment, the mirror is comprised of fragments known to have SNPs on them. The array to identify the SNPs present in the sample may be oligos with mis-match in the position of the SNP or an oligo with extension properties at the site of the SNP. A major advantage of this approach is that, for genome wide analysis of SNPs, one representation is prepared instead of the current method of multiplex PCR where at best 80 percent of the fragments amplify. This allows the simultaneous analysis of many genome wide SNPs.

5.3. Preparation of Microarrays

Microarrays for use in the present invention are known in the art and consist of a surface to which probes can be specifically hybridized or bound, preferably at a known position. Each probe preferably has a different nucleic acid sequence. The position of each probe on the solid surface is preferably known. In one embodiment, the microarray is a high density array, preferably having a density of greater than about 60 different probes per 1 cm².

To manufacture a microarray, DNA probes are attached to a solid support, which may be made from glass, plastic (e.g., polypropylene, nylon), polyacrylamide, nitrocellulose, or other materials, and may be porous or nonporous. A preferred method for attaching the nucleic acids to a surface is by printing on glass plates, as is described generally by Schena et al., 1995, Science 270:467-470. See also DeRisi et al., 1996, Nature Genetics 14:457-460; Shalon et al., 1996, Genome Res. 6:639-645; and Schena et al., 1995, Proc. Natl. Acad. Sci. USA 93:10539-11286.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Light-directed spatially addressable parallel chemical synthesis, Science 251:767-773; Pease et al., 1994, Light-directed oligonucleotide arrays for rapid DNA sequence analysis, Proc. Natl. Acad. Sci. USA 91:5022-5026; Lockhart et al., 1996, Expression monitoring by hybridization to high-density oligonucleotide arrays, Nature Biotech 14:1675; U.S. Pat. Nos. 5,578,832; 5,556,752; and 5,510,270, each of which is incorporated by reference in its entirety for all purposes) or other methods for rapid synthesis and deposition of defined oligonucleotides (Blanchard et al., 1996, High-Density Oligonucleotide arrays, Biosensors & Bioelectronics 11:687-90). When these methods are used, oligonucleotides (e.g., 20-mers) of known sequence are synthesized directly on a surface such as a derivatized glass slide.

Other methods for making microarrays, e.g., by masking (Maskos and Southern, 1992, Nuc. Acids Res. 20:1679-1684), may also be used. Any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989, which is incorporated in its entirety for all purposes), could be used, although, as will be recognized by those of skill in the art, very small arrays will be preferred because hybridization volumes will be smaller. Presynthesized probes can be attached to solid phases by methods known in the art.

5.4. Preparation of Sample Nucleotides

Sample to be hybridized to microarrays can be labeled by any means known to one of skill in the art. The sample may be from any source, including a representation, cDNA, RNA or genomic DNA. In a particular embodiment, the sample is labeled with a fluorescent probe, by, for example, random primer labeling or nick translation. When the sample is a representation, it may be labeled during the PCR step of making the representation by inclusion in the reaction of labeled nucleotides. The fluorescent label may be, for example, a lissamine-conjugated nucleotide or a fluorescein-conjugated nucleotide analog. Sample nucleotides are preferably concentrated after labeling by ultrafiltration.

In a particular embodiment, two differentially labeled samples (e.g., one labeled with lissamine, the other fluorescein) are used.

5.5. Hybridization to Microarrays

Hybridization of a sample to an array encompasses hybridization of the sample, or nucleotides derived from the sample by any method, for example by using the sample as a template for nucleic acid synthesis (e.g., nick translation, random primer reaction, transcription of RNA from represented DNA), or by manipulating the sample (e.g., size fractionation of the sample, gel purified fragments from the sample to the array).

Nucleic acid hybridization and wash conditions are chosen such that the sample DNA specifically binds or specifically hybridizes to its complementary DNA of the array, preferably to a specific array site, wherein its complementary DNA is located, i.e., the sample DNA hybridizes, duplexes or binds to a sequence array site with a complementary DNA probe sequence but does not substantially hybridize to a site with a non-complementary DNA sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., supra, and Chee et al., 1996, Science 274:610-614).

Arrays containing double-stranded probe DNA situated thereon are preferably subjected to denaturing conditions to render the DNA single-stranded prior to contacting with the sample DNA. Arrays containing single-stranded probe DNA (e.g., synthetic oligodeoxyribonucleic acids) need not be denatured prior to contacting with the sample DNA.

Optimal hybridization conditions will depend on the length (e.g., oligomer versus polynucleotide greater than 200 bases) and type (e.g., RNA, DNA) of probe and sample nucleic acids. General parameters for specific (i.e., stringent) hybridization conditions for nucleic acids are described in Sambrook et al., supra, and in Ausubel et al., 1987, Current Protocols in Molecular Biology, Greene Publishing and Wiley-Interscience, New York. When the cDNA microarrays of Schena et al. are used, typical hybridization conditions are hybridization in 5×SSC plus 0.2% SDS at 65° C. for 4 hours followed by washes at 25° C. in low stringency wash buffer (1×SSC plus 0.2% SDS) followed by 10 minutes at 25° C. in high stringency wash buffer (0.1×SSC plus 0.2% SDS) (Shena et al., 1996, Proc. Natl. Acad. Sci. USA 93:10614). Useful hybridization conditions are also provided in, e.g., Tijessen, 1993, Hybridization With Nucleic Acid Probes, Elsevier Science Publishers B.V. and Kricka, 1992, Nonisotopic DNA Probe Techniques, Academic Press San Diego, Calif.

5.6. Detection of Hybridization

Hybridization to the array may be detected by any method known to those of skill in the art. In a particular embodiment, the hybridization of flourescently labeled sample nucleotides is detected by laser scanner. When two different fluorescent labels are used, the scanner is preferably one that is able to detect fluorescence of more than one wavelength, the wavelengths corresponding to that of each fluorescent label, preferably simultaneously or nearly simultaneously.

The invention having been described, the following example is offered by way of illustration and not limitation.

### 6. EXAMPLES

6.1. Generation and Use of Reflections

Materials and Methods

DNAs: The BAC collection is from the Pieter De Jong library. Each pool of BACs contains 1920 different BACs, three pools were made (A, B, C) with 384 overlapping BACs between pool A and B, and pool B and C. The 4992 clones from the library were grown individually in 96-well plates until saturation (approximately for 20 hours) and then pooled. The BAC DNAs were extracted as follows. Each BAC was grown in 25 ml of 2×LB (NaCl, Bacto tryptone, and Yeast Extract) for 24 hours. Bacteria were then pelleted and resuspended in Qiagen P1 buffer, treated with RNase followed by the addition of Qiagen Buffer P2 to lyse the cells. After 1 hour of room temperature incubation, Qiagen buffer P3 was added and placed on ice. After 1 hour samples were filtered through cheesecloth, and DNA precipitated by the addition of isopropanol. After 70% ethanol wash the pellets were air dried briefly and then resuspended in 40 μl of Tris-EDTA.

Probe collections: The collection of cDNAs is the Unigene set available from Research Genetics (Huntsville, Ala.) and an array of 10,000 cDNAs was used in this study.

Microarray experiments: Total human DNA, representations or reflected representations have been labeled according to the protocol described in Lucito, R., West, J., Mishra, B., Reiner, A., Yen, C., Esposito, D., Alexander, J., Wigler, M., and Norton, L. (2000), Genome Res. 10, 1726-1736. Preparation of the probes for arraying and the arraying are also described in Schena et al., 1995 and Derisi et al., 1996. After scanning the arrays with an Axon Genepix4000A, the microarray data analysis was performed using Axon Genepix Pro3.0, S-plus2000 and Spotfire.net desktop5.0.

Sample preparation: Three different methods of preparing the sample were compared.

The first is the preparation of a Sau3AI or BglII representation of the pools of BACs according to the protocol described in Lucito, R., Nakimura, M., West, J. A., Han, Y., Chin, K., Jensen, K., McCombie, R., Gray, J. W., and Wigler, M. (1998), Proc. Natl. Acad. Sci. USA 95, 4487-4492, using IBglII24 and IBglII12 as adaptors (IBglII24: 5′TCA GCA TCG AGA CTG AAC GCA GCA3′, IBglII12: 5′GAT CTG CTG CGT3′from Life Technologies).

The second way of preparing the sample is by making a reflection of a Sau3AI or BglII representation against a pool of printed probes (cDNAs or genomic BglII probes). A complete, restriction endonuclease digestion of the pool of BACs has been performed (Sau3AI or BglII) using 20 Units of enzyme for 1 μg of DNA, overnight at 37° C. After a phenol/extraction, precipitation, the DNA was ligated to IBglII24 adaptor according to the protocol described in Lisitsyn, N. and Wigler, M. (1993), Science 258, 946-951 (Lisitsyn et al., 1993). Then, a representation was prepared with 10 cycles PCR (Lisitsyn et al., 1993). The PCR product is phenol-extracted, and precipitated. 1.5 μg of the representation of the BAC DNA was used to perform a liquid hybridization. The genomic DNA was mixed with 60 ng of the pooled probes from the collection in 10 μl total volume of EE×3 buffer (30 mM EPPS buffer, Sigma, pH 8.0 at 20° C., 3 mM EDTA). The sample was denatured and 1 μl of 5M NaCl added and hybridized for 13 hours at 65° C. A more detailed protocol can be found in (Lisitsyn 1993). The heteroduplexes formed during the hybridization from one strand of the cDNAs and one strand of the BAC DNAs were purified using the Magnashere Magnetic Separation Products from Promega. The Promega kit includes magnetic beads linked to streptavidin. As each cDNA strand has a biotinylated primer, the heteroduplexes mentioned above, as the homoduplexes from the pool of cDNAs will be linked to the beads. An alkaline elution (0.2N NaOH for 2 min, 0.2N HCl for 2 min., at 65° C.) was performed to denature the double strand DNAs after four washes with 0.1×SSC at 65° C. In addition to the biotin on the primer the cDNA pools have dUTP incorporated (See below). To avoid any contamination of mirror the magnetic separation was followed by a Uracil-DNA (UDG, New England Biolabs) treatment (Longo 1990) (10U/DNA eluted (60 ng), for 1 h at 37° C.) followed by a N,N′-Dimethylethylene-diamine treatment (100 mM final for 30 min at 95° C.) (McHugh, 1995) to cut the DNA strands containing dUTP. The treated DNA was then amplified by PCR (Lisitsyn et al., 1993) using IBglII24 as primer and approximately 10 ng of DNA to get the reflected representation. Adaptors are digested and DNA is cleaned according to the procedure described in Lisitsyn et al., 1993.

The third preparation of sample was identical to the second method with the following exceptions. After restriction digestion and ligation of adaptors, the ends of the BAC DNA fragments were filled in (the 30 μl of the ligation are added to 40 Ξl of 5×RDA buffer (Lisitsyn et al., 1993), 16 μl of 4 mM dNTPs (Amersham Pharmacia), 114 μl of distilled water and 7.5U of Taq polymerase (Perkin Elmer), and incubated at 72° C. for 30 min) and no amplification was performed before the liquid hybridization.

Preparation of the pool of probes (cDNAs or BglII probes): The pools of probes or “mirror” are made from the stock of plates that are used for printing the microarrays. Each pool has been purified using the Qiaquick purification kit from Qiagen. 20 ng of DNA is then amplified by PCR for 20 cycles (1 min 94° C., 1 min 55° C., 2 min 72° C.) using biotinylated M13 universal primers at 62 pM per μl (for the cDNAs: M13rev 5′GTG AGC GGA TAA CAA TTT CAC ACA GGA AAC AGC, M13fwd 5′CTG CAA GGC GAT TAA GTT GGG TAA C and for the BglII probes: M13rev 5′GGA AAC AGC TAT GAC CAT GA, M13fwd 5′TTG TAA AAC GAC GGC CAG TG), 16 μl of 4 mM dNTPs containing 20% of dUTP, 40 μl of 5×RDA (Lisitsyn 1993) buffer, 120 l of distilled water and 7.5U of Taq polymerase. The PCR product is then purified again using the Qiaquick purification kit.

Validation of the microarray experiments by PCR: From the clones on the cDNA array, acession numbers for specific genes have been selected informatically from a Spotfire.net desktop5.0 scatter plot. The cDNA sequences were then retrieved from Genbank NCBI and a Blast search (Altschul et al., 1990, J. of Molec. Biol., 215:403-410) was performed against BAC sequences. Blast results were informatically analyzed using a visual Basic program in order to design primers from the genomic sequences matching the cDNAs, free of internal intron and spanning a small Sau3AI fragment.

The designed primers were used to amplify by PCR total human genomic DNA, each pool of BACs and the pool of cDNAs (25 ng of each DNA, 10 μl of 5×RDA (Lisitsyn 1993) buffer, 2.5 μl of 4 mM dNTPs, 1 μl of a 10 fold dilution of 1 mM primer solution (for each primer), qsp H₂O 50 μl and 2.5U of Taq polymerase, for 30 cycles at 1 min 94° C. and 3 min 72° C., 10 min 72° C.

Results

The concept of a reflection of DNA is presented graphically in FIG. 1. In this study, we demonstrate the usefulness of reflections of DNA for the mapping of arrayed cDNAs to the appropriate pool of BACs. Mapping is the process by which elements of a genome are placed in an ordered relation to each other. Therefore, any mapping consists of at least two operations: 1) the isolation from the genome of well defined sub-genomic elements, for example a collection of BACs or probes; and 2) establishment of a relationship between probes and BACs by, for example, hybridization. An ordered relationship between probes and BACs can be established by hybridization. Two BACs overlap if they hybridize to a common probe, and two probes are neighbors if they hybridize to a common BAC. If the assignment of a probe to a BAC can be accomplished by microarray hybridization, then microarrays can be used to map genomes.

DNA from each pool of BACs was used to make standard high complexity SAU3AI representations, and also to make two reflected representations. For the reflections, the mirrors consisted of pooled cDNA probes. Two pools of cDNAs were used as mirrors, each consisting of non-overlapping subsets of 500 each, representing the odd and even rows of probes from the microarray prints. Each type of representation was prepared independently twice, as replicas, and hybridized to microarrays along with total human DNA as a common denominator. The Cy5/Cy3 symmetric ratios (representing the ratio of BAC DNA to total human DNA hybridized to each point of the array) of replica hybridizations are plotted in FIG. 2.

The FIG. 2A displays the results of parallel hybridizations with high complexity representations of the BAC pool B, and FIG. 2B displays the results of hybridizations of the reflection of BAC pool B using cDNAs from the odd rows of the array as the mirror. The results of parallel hybridizations of replicas are roughly reproducible, as can be seen by the expected diagonal distribution of the ratios. From FIG. 2, it is concluded that higher ratios are observed when the sample is reflected, indicating that reflection enhances the signal of specific hybridization to the array.

Not all ratios are elevated following hybridization to reflected samples. In fact, the increase in ratios is only observed in the subset of cDNAs used as the mirror. This is illustrated in FIG. 3, which shows the symmetric ratios obtained for probes by odd and even row from one series of experiments. Only the ratios for cDNAs from odd rows show upward scatter (in FIG. 3D). It is the DNA from the odd rows that was used as the mirror. Even within the odd-rowed set, most probes will not hybridize with the BAC pools, and the ratio for these probes would not be expected to change. The even rowed cDNAs were not used as a mirror, and for these, the ratios are in fact somewhat compacted, resulting from lower signal-to-noise.

With reflective representations, probes can be readily and unambiguously assigned to a specific BAC pool (see FIG. 4). In FIG. 4, features exhibiting a high ratio with respect to a single pool (i.e., are found along either the X or the Y axis, not along the diagonal running from lower left to the upper right) are candidates for assignment to that pool. To identify such probes, probes were selected that exhibited a Cy5/Cy3 ratio in excess of 6 from each experiment wherein a reflected BAC pool and total human DNA were compared, and then probes that were common to more than one pool were deleted. For pools reflected against the odd rowed probes, a total of 172 probes out of 5000 received unique assignments in this manner.

TABLE 1
Validation of reflected genes

| | | Pairs of | |
|---|---|---|---|
| | Clones selected | primers | Validated genes |
| | from Spotfire | tested | by PCR (%) |
| Unique clones from pool A | 62 | 14 | 13 (93) |
| Unique clones from pool B | 53 | 9 | 8 (89) |
| Unique clones from pool C | 57 | 11 | 10 (91) |

In Table 1, the first column is the number of genes picked up from FIG. 4 having an elevated ratio for BAC pool A, B and C. For each of these genes, a number of primer pairs have been designed (second column) and PCR performed as explained in the legend to FIG. 5. The third column gives the PCR results as the number and percentage of cDNAs that could be assigned to a specific BAC pool.

FIG. 5 depicts the confirmatory results obtained for probes assigned to pool A. As can be seen, for each pair of probes, only DNA from pool A (lane A), genomic DNA (Lane G), and the mirror cDNA (lane M) allow for the production of a specific PCR product, indicating that the sequence amplified by the pair of probes exists only in pool A, and not in pool B or C, and can be found, as expected, in the cDNAs of the array and in total genomic DNA.

The foregoing specification is considered to be sufficient to enable one skilled in the art to broadly practice the invention. Indeed, various modifications of the above-described methods for biochemistry, organic chemistry, medicine or related fields are intended to be within the scope of the following claims. All patents, patent applications, and publications cited are incorporated herein by reference in their entirety for all purposes.

What is claimed is:

1. A method of producing a reflection of DNA fragments, comprising:
(a) contacting a first collection of DNA fragments with a second collection of DNA fragments under conditions such that hybridization between the first collection of fragments and the second collection of fragments can occur, thereby forming heteroduplex double stranded DNA fragments having a first strand of DNA from the first collection of DNA fragments and a second strand of DNA from the second collection of DNA fragments; wherein the second collection of DNA fragments is biotinylated and has at least 10% of the thymidine residues present replaced with uracil;
(b) contacting the hybridized biotinylated fragments of step (a) with immobilized streptavidin under conditions such that the biotinylated fragments are bound by the streptavidin;
(c) rinsing away any DNA fragments not bound to the streptavidin, thereby purifying the biotinylated fragments;
(d) releasing the biotinylated fragments from the streptavidin;
(e) specifically degrading the strands of DNA from the second collection of fragments by contacting the product of step (d) with UDG followed by N,N′-Dimethylethylene-diamine; and
(f) synthesizing double stranded DNA from the first strand using the first strand as template.

2. A reflection of DNA prepared according to the method of claim 1.

3. A method of hybridizing nucleic acids from one or more samples to an array of probe DNA immobilized on a surface of a solid phase comprising: contacting said array, containing or suspected of containing sequences complementary to nucleic acids from one or more samples, with nucleic acids from one or more samples, under conditions such that hybridization between the nucleic acids and probe DNA can occur, wherein the one or more sample nucleic acids are or are derived from a reflection prepared according to the method of claim 1.
